(19) United States
(12) Patent Application Publication
(10) Pub. No.: US 2022/0323490 A1
(43) Pub. Date: Oct. 13, 2022

(54) PREVENTION OF DISEASES IN HONEYBEES

(71) Applicant: Arkema Inc., King of Prussia, PA (US)

(72) Inventor: Christian G. Becker, King of Prussia, PA (US)

(21) Appl. No.: 17/641,887

(22) PCT Filed: May 13, 2020
(86) PCT No.: PCT/US2020/032554

§ 371 Date: Mar. 10, 2022

Related U.S. Application Data
(63) Continuation of application No. 62/851,688, filed on May 23, 2019.

(51) Int. Cl. A61K 33/40; A61P 31/00; A01K 53/00; A01K 51/00; A23K 10/30

(52) U.S. Cl. CPC A61K 33/40; A61P 31/00; A01K 53/00; A01K 51/00; A23K 10/30

(57) ABSTRACT

Feeding bees a composition containing at least one oxidizing agent (e.g., peroxide), optionally also containing other components such as an oxidizing agent activator or a viscosifying agent, to treat against, reduce and/or limit the spread of bacterial, virus, and fungi infestation in bees and/or beehives.

### FIELD OF THE INVENTION

The present invention pertains to methods for the prevention and/or treatment of unwanted pathogens or diseases in honeybees via the addition of hydrogen peroxide or peroxygen source component to materials fed to bees. The feeding of hydrogen peroxide to bees provides for spread of the hydrogen peroxide throughout the hive via trophallaxis to act as a bee disease prophylaxis.

### BACKGROUND OF THE INVENTION

There are numerous diseases threatening honeybees and honey production. These diseases arise from sources such as bacteria, fungi, viruses, fungi protozoa and mites.

The honeybee, Apis mellifera, plays a vital role in agriculture by assisting in the pollination of a wide variety of crops. Unfortunately, the health and vigor of honeybees colonies are threatened by numerous parasites and pathogens, including viruses, bacteria, protozoa, and mites. As a relatively homogeneous community of thousands of related individuals, one would expect a honeybee colony to be highly susceptible to the transmission of disease organisms. The high density of individuals within the colony, their close physical contact (through casual contact, communication and mutual grooming) and the trophallactic interchange of food and glandular substances all provide numerous and diverse opportunities for pathogen transmission. Furthermore, the beehive, with its relative constant temperature and moderate humidity, provides an ideal environment for pathogens to grow.

According to the United States Department of Agriculture, Animal and Plant Health Inspection Service, Survey of Honey Bee Pests and Diseases, Apr. 7, 2017, honeybee health decline has been documented for years. In recent years, winter losses have been unsustainably high, ranging from 22% to 36% nationally. These rates of loss threaten the viability of beekeeping operations and the production of crops dependent on bees for pollination as well as honey production.

In addition to pests and parasites, honeybees are also subject to a multitude of diseases: bacterial diseases such as American foulbrood, European foulbrood, fungal diseases such as Chalkbrood; Stonebrood; Nosema, and viral diseases such as Cripaviridae—Chronic bee paralysis virus, Dicistroviridae—Acute bee paralysis virus, Israeli acute paralysis virus, Kashmir bee virus, Black queen cell virus; Cloudy wing virus; Sacbrood virus; Iflaviridae—Deformed wing virus, Kakugo virus; Iridoviridae—Invertebrate iridescent virus type 6, Secoviridae—Tobacco ringspot virus; Lake Sinai virus.

American foulbrood (AFB) is one of the most virulent brood diseases known in honeybees. The disease is caused by the spore forming bacterium Paenibacillus larvae. American foulbrood spores are extremely resistant to desiccation and can remain viable for more than 40 years in honey and beekeeping equipment. Each dead larva may contain as many as 100 million spores. Because the spores can remain viable for years, many countries require bee colonies with AFB to be burned. Other countries (e.g., USA, Canada, and Argentina) allow the use of antibiotics to keep the disease in control.

A spray composition described in U.S. Pat. No. 1,242,770 comprises a composition made of water, salt, carbolic acid, hydrogen peroxide and sulfuric acid, is sprayed directly in the beehive as a remedy for European foulbrood.

Nosemosis is by far the most widespread and the most damaging adult bee disease. Infections are acquired by the uptake of spores during feeding or grooming. Nosema apis is a microsporidian, a small, unicellular parasite recently reclassified as a fungus that mainly affects honey bees. It causes nosemosis, also called nosema, which is the most common and widespread of adult honeybee diseases. Actually, nosema is so widespread that it is presumed that every colony has some infected bees. It is often treated with antibiotics.

Treating with antibiotics in order to prevent or save colonies from diseases is obviously an issue as it introduces long lasting chemical residues in the beehive, which in turns can end up in honey and wax frames. In the EU, honeybees are classified as food producing animals. Accordingly, a maximum residue limit (MRL) for honey must be met before a marketing authorization can be granted. Therefore, in principle, only medicinal products, which do not result in residues in honey, could be authorized.

This is obviously a problem for trade as some countries are banning the use of antibiotics and some countries authorize antibiotics only under specific circumstances. It is also very troubling that antibiotics could be present in beeswax and honey.

Non-limiting examples of antibiotics or chemotherapeutics used in various countries are Streptomycin, Tetracyclines, Sulfonamides, Erythromycin, Tylosin, Lincomycin, Enrofloxacin, Ciprofloxacin, Trimethoprim, Metronidazole, Choramphenicol, Nitrofurans. [Antimicrobials in beekeeping—W. Reybroeck, E. Daeseleire, H. De Brabander, L. Herman, Veterinary Microbiology 158 (2012) 1-11].

Risks related to the use of antibiotics for the control of honeybee diseases are persistence of the infection, reappearance of the disease and honey contamination. It would therefore be advantageous to be able to treat against bacteria, viruses, fungi, protozoa and mites using a product that would not leave any chemical residues. The present invention is directed towards the use of oxidizing agents such as hydrogen peroxide to provide both the sanitation effect and zero residue aspect of the treatment.

Disinfection of beehives and beehive equipment using non-antibiotic products was proposed and disclosed in prior art. United States Patent Publication No. 2009/0104288 discloses the use of a hop derivative to treat beehives. U.S. Pat. No. 6,096,350 discloses the treatment of honeybee by applying an effective amount of an aqueous composition comprising a protic acid and a chlorite ion. United States Patent Publication No. 2009/01182143 discloses increasing the tolerance of bees to disease by feeding bees an effective amount of the nucleic acid agent comprising a nucleic acid sequence down regulating expression of a gene product of a bee pathogen. While all these methods have merit, they are either complicated, expensive or difficult to implement. In addition, residues of the solution added to the beehives are expected to be present after treatment.

Contaminants can reach the raw materials of bee products when transported into the beehives by the bees after foraging pollen, nectar and water from plants that have been sprayed with pesticides (or added to the vegetation by other means such as seed treatments) by farmers, agrochemical professionals, gardeners and the likes in order to control a variety of agricultural pests that can damage crops.

However, the most important contaminants are probably the substances used in the control of honeybee pests. There are many bee pests, parasites and diseases: Varroa mites (Varroa destructor or Varroa jacobsoni), Acarine (tracheal) mites (Acarapis woodi), small hive beetles (Aethina tumida), wax moths (Galleria mellonella), tropilaelaps (Tropilaelaps clareae and T. mercedesae), nosema disease, American and European foulbrood, and so forth.

At present, one of the most important pests worldwide is Varroa destructor. Varroa destructor (varroa mite) is an external parasitic mite that attacks the honeybees Apis cerana and Apis mellifera. The disease caused by the mites is called varroosis. Acaricides used in the control of Varroa destructor are a major source of pollution because they often involved slow release products and must be present in the beehives for a period up to 45 days to be effective and prevent re-infestation [The concentration effect of selected acaricides present in beeswax foundation on the survival of Apis mellifera colonies, S. Medici, A. Castro, E. Sralo, J. Marioli, M. Eguaras, Journal of Apicultural Research 51(2):164-168 April 2012].

Both the unintentional and the intentional exposures of honeybees (and other pollinators) to pesticides have resulted in pesticide residues being detected in the beehives, bees, pollen, honey and especially beeswax (brood nest wax and beeswax foundation). The roles of these pesticides and their residues in hive products may have played a role in colony collapse disorder (CCD) and in other colony problems observed in the last several years [Pesticides and honey bee toxicity—USA, R. M. Johnson, M. D. Ellis, C. A. Mullin, M. Frazier, Apidologie, 41, issue 3, (2010), 312-331]. Colony collapse disorder causes significant economic losses because many agricultural crops worldwide are pollinated by western honeybees. Although the causes for bee declines are diverse and not always well understood, honeybee exposure to pesticides can have a severe impact.

Chronic exposure to pesticides have long been suspected as a potential cause of honeybee declines. In a recently published study [High Levels of Miticides and Agrochemicals in North America Apiaries: Implications for Honey Bee Health, C. Mullin, M. Frazier, J. Frazier, S. Ashcraft, R. Simonds, D. vanEngelsdorp, J. Pettis, PLoS ONE, march 2010, vol. 5, issue 3, e9754], most comb and foundation waxes sampled were found contaminated with 87 diverse pesticides and metabolites, with up to 39 different detections in a single sample, averaging 8 different pesticide residues each. The most frequent detections were the in-hive acaricides fluvalinate and coumaphos (two very common miticides), the organophosphate pesticide chlorpyriphos and chlorothalonil, a widely used fungicide.

Examples of typical miticide commercial products used in beehives are fluvalinate (Apistan® anti-varroa mite strips), amitraz (Apivar®), or coumaphos (Checkmite+® beehive pest control strip). A complete list of such chemicals can be found on the Environmental Protection Agency (EPA) registered pesticide products list approved specifically for use in beehives and on the list of pesticides approved for application on the various life stages of crops.

As used herein, the term “pesticide” includes all of the following: herbicides, insecticides, insect growth regulators, nematicides, termiticides, molluscicides, piscicides, avicides, rodenticides, predacides, bactericides, insect repellents, animal repellents, antimicrobials, fungicides, disinfectants (antimicrobials), and sanitizers.

Most pesticides, fungicides and acarides are lipophilic or fat soluble, non-volatile and persistent, and thus easily accumulate in the beeswax. It is common beekeeping practice to recycle beeswax almost continuously. As a consequence, pesticides can be accumulated over a period of several years creating an unhealthy toxic environment for the bees and brood. Additionally, pesticides often resist degradation at the wax melting temperatures used during wax processing. It has been shown that a purification process involving melting the beeswax in boiling water does not substantially modify the initial content of the lipophilic contaminants in beeswax when these are present in relatively high concentrations [Residues of Organic contaminants in beeswax, J. J. Jimenez, J. L. Bernal, M. J. del Nozal, M. T. Martin, Eur. J. Lipid Sci. Technol. 107 (2005) 896-902].

### SUMMARY OF THE INVENTION

The novel concept presented in the present invention relates to the use of trophallaxis to propagate peroxide chemicals capable of treating, reducing or limiting the spreading of unwanted pathogens in bee colonies. It was found for example that hydrogen peroxide is stable in the presence of sugar solutions. The addition of hydrogen peroxide to a sugar solution fed to bees helps spread the peroxide within the entire colony and acts as a bee disease prophylaxis.

Aspect 1: A method of treating, preventing or reducing pathogen infections in bees and beehives comprising, consisting essentially of or consisting of feeding bees a composition comprised of, consisting essentially of or consisting of at least one oxidizing agent.

Aspect 2: The method of Aspect 1, wherein the composition is comprised of at least one peroxide as an oxidizing agent.

Aspect 3: The method of any of Aspects 1-2, wherein the composition comprises, consists essentially of or consists of at least one peroxide selected from the group consisting of hydrogen peroxide, peroxyacids, peroxycarbonates, urea hydrogen peroxide, perborate compounds, and combinations thereof.

Aspect 4: The method of Aspects 1 to 3, wherein the peroxide composition is comprised of from about 0.001% to about 50%, from about 0.01% to about 5%, or from 0.1% to about 3% by weight peroxide.

Aspect 5: The method of Aspects 1 to 4, wherein the composition is additionally comprised of at least one peroxide activator.

Aspect 6: The method of Aspect 5, wherein the at least one peroxide activator comprises, consists essentially of or consists of at least one peroxide activator selected from the group consisting of metal-containing peroxide activators, carbonate salts and combinations thereof.

Aspect 7: The method of any of Aspects 5 to 6, wherein the composition is comprised of from about 0.001% to about 20% by weight or from about 0.001% to about 5% by weight peroxide activator.

Aspect 8: The method of any of Aspects 1 to 7, wherein the composition further comprises at least one viscosifying agent and/or gelling agent.

Aspect 9: The method of any of Aspects 1 to 8, wherein the composition is comprised of from about 0.01% to about 10.0% by weight or from about 0.1% to about 5.0% by weight in total of viscosifying agent and/or gelling agent

Aspect 10: The method of any of Aspects 1 to 9 wherein the pathogens are selected from the group consisting of the American Foulbrood (AFB) disease, Chalkbrood disease and Nosema disease.

Aspect 11: The method of any of Aspects 1 to 10 wherein the pathogens are selected from the group consisting of American foulbrood (AFB), and European foulbrood (EFB); fungal diseases selected from the group consisting of Chalkbrood, Stonebrood, and Nosema; and viral diseases selected from the group consisting of Cripaviridae, Chronic bee paralysis virus, Dicistroviridae, Acute bee paralysis virus, Israeli acute paralysis virus, Kashmir bee virus, Black queen cell virus, Cloudy wing virus, Sacbrood virus; Iflaviridae—Deformed wing virus, Kakugo virus; Iridoviridae—Invertebrate iridescent virus type 6, Secoviridae—Tobacco ringspot virus, and Lake Sinai virus.

Aspect 12: The method of any of Aspects 1 to 10 wherein the pathogens are bacteria selected from the group consisting of Melissococcus plutonius, Paenibacillus larvae, Spiroplasma apis, S. melliferum, Pseudomonas aeruginosa, Achromobacter euridice, Enterococcus faecalis, Paenibacillus alvei, and Brevibacillus laterosporus.

Aspect 13: The method of any of Aspects 1 to 10 wherein the pathogens are fungi selected from the group consisting of Nosema apis, Nosema ceranae, Ascosphaera apis, and Aspergillus spp.

Aspect 14: The method of any of Aspects 1 to 9 wherein the pathogen responsible for a disease is a virus.

Aspect 15: The method of any of Aspects 1 to 10 wherein the pathogens are viruses selected from the group consisting of Israeli acute paralysis virus, acute bee paralysis virus, Kashmir bee virus, black queen cell virus, deformed wing virus/Kakugo virus, Varroa destructor virus, sacbrood virus slow bee paralysis virus, chronic bee paralysis virus and Lake Sinai virus.

Aspect 16: The method of any of Aspects 1 to 10 wherein the pathogens are the bacteria spores, fungi spores or yeast.

Aspect 17: The method of Aspects 1 to 10 wherein the pathogen is selected from the group consisting of Varroa mites (Varroa destructor or Varroa jacobsoni), Acarine (tracheal) mites (Acarapis woodi), small hive beetles (Aethina tumida), wax moths (Galleria mellonella), and tropilaelaps (Tropilaelaps clareae and T. mercedesae)

Aspect 18: The method of any of Aspects 1 to 17 wherein said composition is a bee-ingestible composition in liquid form.

Aspect 19: The method of any of Aspects 1 to 17 wherein said composition is a bee-ingestible composition in solid form.

Aspect 20: The method of any of Aspects 1 to 19 wherein said composition contains protein.

Aspect 21: The method of any of Aspects 1 to 20 wherein said composition is based on pollen and/or soy patties.

Aspect 22: The method of any of Aspects 1 to 21 wherein said composition is a bee-ingestible composition based on sucrose solution.

Aspect 23: The method of any of Aspects 1 to 22 wherein said composition is a bee-ingestible composition based on corn syrup solution.

Aspect 24: The method of any of Aspects 1 to 23 wherein said composition is a bee-ingestible composition that further comprises a carbohydrate or sugar supplement.

Aspect 25: The method of any of Aspects 1 to 24 wherein the composition is administered to the bees via a device selected from the group consisting of strip, controlled release strip, tablet, reservoir, polymer disc, evaporation device, fiber, tube, polymeric block, membrane, pellet, and microcapillary comprising the composition.

### DETAILED DESCRIPTION OF CERTAIN EMBODIMENTS OF THE INVENTION

Dispensing of hydrogen peroxide to the hive can be done by introducing the compound in the composition used in supplemental feeding of honeybees, which is common practice. Honeybees can be fed various foodstuffs to supplement inadequate supplies of pollen or honey. In early spring before pollen and nectar are available or at other times of the year when these materials are in short supply, supplementary feeding may help the colony survive or make it more populous and productive. In practice, beekeepers feed their bees supplemental foods to develop and maintain colonies with optimum populations. The hydrogen peroxide compound is added at the desired concentration to the composition to be fed to the bees. The composition can be a liquid, gel or solid. As an alternative, the composition can be administered to the bees by being dispensed from a variety of forms or devices. The devices can include a strip, controlled release strip, tablet, reservoir, polymer disc, evaporation device, fiber, tube, polymeric block, membrane, pellet, and microcapillary.

A common foodstuff for honeybees is a mixture of water and sugar, a sucrose solution. A typical water/sugar mixture comprises a ratio of one part of sugar to one part of water, measured by weight (known as 1:1). A denser syrup of two parts of sugar to one part of water (known as 2:1) may also be used. Generally, 1:1 syrup is used to supplement honey stores, stimulate colonies to rear brood and encourage drawing of comb foundation particularly in spring. The stronger syrup is used to provide food when honey stores in the hive are low. Measuring the sugar and water by weight or volume is all right, as there is no need to be 100% exact about the sugar concentration. Similar foodstuff solutions for bees can be based upon corn syrup.

The composition of the present invention can also be a component of a solid bee foodstuff such as pollen and/or soy patties, with or without added proteins, carbohydrates or sugar supplements.

The composition used with the present invention includes at least one oxidizing agent, which may be any organic or inorganic compound capable of treating against any bacterial, viruses, fungi, and protozoa infestation. Oxidizing agents can be selected from a variety of peroxides. For example inorganic and organic peroxides such as but not limited to hydrogen peroxide or peroxide generating compounds such as percarboxylic acids (e.g., peracetic acid), peroxycarbonates, urea hydrogen peroxide, perborate compounds, as well as similar compounds and/or a combination of such compounds. The concentration of oxidizing agent in the composition may be between 0.001 and 50%, or between 0.01 and 5%, or between 0.1 and 3% by weight in a foodstuff, preferably a sucrose solution.

The composition can be gelled or otherwise increased in viscosity for better handling of the composition using at least one viscosifying agent, which may be an inorganic viscosifying agent or an organic viscosifying agent or a combination of viscosifying agents. Examples of suitable viscosifying agents include, for example, modified silicas (e.g., the silicas sold by Evonik under the brand names Aerosil® and Sipernat®), high molecular weight crosslinked polyacrylic acid polymers (e.g., the polymers sold by Lubrizol under the brand name Carbopol®), xanthan gums (e.g., the xanthan gums sold by CP Kelco under the brand name Kelzan®) and other such gums (guar gums, alginates and the like), polyol and polyether glycol compounds such as glycerol, polyethylene glycol, polypropylene glycol, and all other viscosifying or gelling agents that are compatible with the other components of the composition and preferably nontoxic to bees and environmentally friendly (e.g., non-persistent and/or biodegradable). The viscosifying agent and/or gelling agent may, in certain embodiments of the invention, act as a thixotropic agent. In various embodiments of the invention, the viscosifying or gelling agent concentration in the composition is between 0.01% and 20% by weight or between 0.1% and 5% by weight.

The order and manner in which the above-described components of the composition are combined and formulated are not believed to be particularly limited. Methods and techniques known in the art may be adapted and modified as appropriate, depending upon the types and relative amounts of the components selected for use. In certain embodiments, the composition may be a one-part formulation, having sufficient physical and chemical stability to permit storage at normal conditions (e.g., in drums, tanks or other containers at room temperature) for extended periods of time. Such formulations may then be directly utilized in accordance with any of the procedures described herein. In other embodiments, the composition may be provided as a multi-part formulation, particularly where certain components of the composition are reactive with each other and it is desired to avoid such reaction until the composition is to be fed to bees. For example, where the composition comprises both a peroxide and a peroxide activator, the peroxide activator may undesirably react prematurely with the peroxide or otherwise transform the peroxide such that the effectiveness of the composition. In such cases, the composition may comprise two parts which are stored separately, wherein one part comprises the peroxide (and optionally one or more components of the composition other than the peroxide activator) and a second part comprises the peroxide activator (and optionally one or more components of the composition other than the peroxide). The two parts are then combined, in the desired proportions, to obtain the composition, either shortly before use.

The composition can be activated by the presence of an activator for the oxidizing agent, i.e., a substance that assists in catalyzing or otherwise promoting disinfecting against any bacterial, viruses, fungi, protozoa and mite infestation. For example, the activator may convert the oxidizing agent into a more reactive substance, e.g., a substance better able to disinfect against any bacterial, viruses, fungi, protozoa and mite infestation. In certain embodiments of the invention, the oxidizing agent formulated into the composition may be regarded as an oxidizing agent precursor, which by itself has little or no impact but which is transformed in situ to a reactive oxidizing agent through interaction with an activator as described herein. Post-addition of an activator to a composition in accordance with the present invention may be practiced. The activator may be added to the composition right before use of the composition. Activators can be, for example, metal-containing substances such iron oxide (Fenton reaction), carbonate compounds (e.g., salts of carbonic acid such as potassium or sodium bicarbonate), a more complex activator such as Fe-TAML (tetra-amido macrocyclic ligand) developed at Carnegie Mellon or any activator or combination of activators that are known to induce the formation of free radical compounds under various conditions (including the use of UV light). The concentration of activator in the composition may be, for example, between 0.001% and 20% by weight or between 0.001% and 5% by weight.

Within this specification, embodiments have been described in a way which enables a clear and concise specification to be written, but it is intended and will be appreciated that embodiments may be variously combined or separated without departing from the invention. For example, it will be appreciated that all preferred features described herein are applicable to all aspects of the invention described herein.

In some embodiments, the invention herein can be construed as excluding any element or process step that does not materially affect the basic and novel characteristics of the composition or the method for using the composition. Additionally, in some embodiments, the invention can be construed as excluding any element or process step not specified herein.

### EXAMPLES

### Example 1: Stability of Hydrogen Peroxide in Sugar Solution

Hydrogen peroxide, Peroxal 50% CLG (available from Arkema Inc.) was used to create concentrations of 0.1% and 1.0% hydrogen peroxide in 50% (w/w) sugar (sucrose) solution. Experiments were done at ambient temperature. No degradation of the hydrogen peroxide was observed over 7 weeks.

TABLE 1
Hydrogen peroxide stability in sugar solution

| H2O2 | Jan. 10, 2017 | Jan. 20, 2017 | Feb. 6, 2017 | Mar. 2, 2017 |
|---|---|---|---|---|
| Conc. | (time 0) | (10 days) | (27 days) | (51 days) |
| 0.1% | 0.134 | 0.134 | 0.134 | 0.130 |
| 1.0% | 1.077 | 1.089 | 1.058 | 1.089 |

The data in Table 1 shows that hydrogen peroxide is stable in a sugar solution that could be used as a bee food.

### Example 2: Determination of the Oral Toxicity of Formulated Hydrogen Peroxide for the Honeybee (Apis mellifera)

The twenty-four and forty-eight hour oral lethal concentration (LC₅₀) of formulated hydrogen peroxide (HP) was determined for one-week and two-week old honeybee (Apis mellifera) workers fed a series of solutions of HP diluted in 2 Molar (M) sucrose.

Five hydrogen peroxide (HP) solutions at 4.7, 3.7, 2.7, 1.7, 0.7 M HP in 2 Molar (M) sucrose, and a 0.00 M HP 2M sucrose solution were prepared using 35% food grade hydrogen peroxide, molecular grade sucrose (Sigma Aldrich, USA), and molecular grade water (Sigma Aldrich, USA).

Approximately 200 bees were collected into several wooden cages, supplied with ad libitum HP-free 2M sucrose solution, water, and BeePro® supplemented pollen patty. Experimental trials commenced at seven and fourteen days after newly emerged honeybees were placed in cages.

Non-adjusted mortality data (mean±standard error (se)) are provided in Table 2.

TABLE 2
Mean (±standard error) mortality of one-week and two-week
old honeybees after feeding on hydrogen peroxide-2M sucrose
solutions for either 24 or 48 hours.
Mean (±se) Mortality

| Concentration | Week 1 | Week 1 | Week 2 | Week 2 |
|---|---|---|---|---|
| H₂O₂ (Molar) | 24 Hour | 48 Hour | 24 Hour | 48 Hour |
| 0 | 0.75 (0.16) | 0.87 (0.40) | 1.13 (0.30) | 1.38 (0.53) |
| 0.7 | 3.38 (1.38) | 9.63 (2.23) | 7.00 (2.19) | 12.75 (1.96) |
| 1.7 | 10.25 (1.42) | 18.13 (1.06) | 12.00 (2.32) | 16.25 (1.50) |
| 2.7 | 15.63 (1.19) | 19.88 (1.63) | 14.13 (2.12) | 19.50 (1.00) |
| 3.7 | 15.25 (1.70) | 21.50 (0.96) | 16.63 (1.92) | 20.75 (0.53) |
| 4.7 | 15.75 (1.53) | 21.38 (0.99) | 17.88 (1.51) | 20.13 (1.36) |

Table 3 gives LC₅₀ values for each assessment point predicted from Probit analyses using login concentrations, LC₅₀ hydrogen peroxide concentrations calculated from predicted values, and corresponding percent hydrogen peroxide (using MW=34 g/mol for H₂O₂, and p=1.25 g/ml for 2M sucrose solution).

The twenty-four hour mortality was significantly lower than the 48-hour mortality for both the one-week (Z=3.41, P<0.001) and two-week old honeybees (Z=2.57, P=0.010) (Table 2). However, mortality did not differ significantly between one-week and two-week old honeybees at either the 24 or 48-hour assessment points.

The twenty-four hour LC₅₀ values were greater than the 48-hour values for both the one-week and two-week old honeybees (Table 3). This was likely due to HP-free sucrose solution remaining in their crops (social stomach of honeybees) even after six hours of starvation prior to beginning trials. Honeybee workers have a high metabolic rate, but also have the ability to share food (via trophallaxis) among nest mates. After forty-eight hours, however, all HP-free sucrose solution was depleted from worker crops and honeybees then fed directly on HP-containing sucrose solutions.

TABLE 3
Mean (±standard error) log₁₀ lethal concentrations of
hydrogen peroxide resulting in 50% mortality (LC₅₀) of one-
week and two-week old honeybees feeding on hydrogen
peroxide-2M sucrose solutions for 24 or 48 hours. Predicted
LC₅₀ values in Molar hydrogen peroxide were calculated by
taking the inverse of log₁₀ data.

| | Predicted LC₅₀ | Predicted LC₅₀ | Predicted LC₅₀ |
|---|---|---|---|
| | Log₁₀ Molar (±se) | (Molar) | (Percent) |
| Week 1-24 Hour | 0.29 (0.02) | 1.95 (0.09) | 5.30 (0.25) |
| Week 1-48 Hour | −0.10 (0.03) | 0.79 (0.05) | 2.15 (0.14) |
| Week 2-24 Hour | 0.15 (0.03) | 1.41 (0.10) | 3.84 (0.28) |
| Week 2-48 Hour | −0.25 (0.06) | 0.56 (0.08) | 1.52 (0.22) |

The LC₅₀ values of two-week old honeybees were slightly lower than those of one-week old honeybees for both the 24 and 48-hour assessment points (Table 2). This is likely due to an increase in sucrose consumption of honeybees as they age. Young adult (nurse) honey bees consume more protein than carbohydrates for the first week post-emergence, but then rapidly increase sucrose consumption after this point.

The no observed effect concentration (NOEC) could not be determined from the data; honey bee mortality observed for all tested concentrations were significantly greater than mortality of honeybees in control group (Wilcoxon multiple comparisons: χ²=113.5; df=5; P<0.001). Following this, an EC₅ was calculated by solving the cubic (sigmoidal) model for ‘x’ given an intercept term constrained to zero (concentration) and a ‘y’ value of 0.05. Model solutions and calculated EC₅ (as % H₂O₂) are provided in Table 4.

TABLE 4
Solutions to cubic models for ECx estimations. Values of X₃
(bold) are best estimates of EC₅ concentration (in Molar
hydrogen peroxide), and were used to calculate EC₅ in
percent H₂O₂.
Solutions for ‘x’ (Molar)

| Assessment point | X₁ | X₂ | X₃ | EC₅ (Percent) |
|---|---|---|---|---|
| Week 1-24 Hour | 2.78 | −5.36 | 0.21 | 0.57 |
| Week 1-48 Hour | 2.69 | −4.58 | 0.15 | 0.41 |
| Week 2-24 Hour | 3.23 | −5.03 | 0.20 | 0.54 |
| Week 2-48 Hour | 2.61 | −4.20 | 0.15 | 0.41 |

Although the invention is illustrated and described herein with reference to specific embodiments, the invention is not intended to be limited to the details shown. Rather, various modifications may be made in the details within the scope and range of equivalents of the claims and without departing from the invention.

1. A method of reducing pathogen infections in bees and beehives comprising of feeding bees a composition comprised of at least one oxidizing agent.

2. The method of claim 1, wherein the composition is comprised of at least one peroxide as an oxidizing agent.

3. The method of claim 1, wherein the composition comprises at least one peroxide selected from the group consisting of hydrogen peroxide, peroxyacids, peroxycarbonates, urea hydrogen peroxide, perborate compounds, and combinations thereof.

4. The method of claim 2, wherein the peroxide composition is comprised of from about 0.001% to about 50%, from about 0.01% to about 5%, or from 0.1% to about 3% by weight peroxide.

5. The method of claim 1, wherein the composition is additionally comprised of at least one peroxide activator.

6. The method of claim 5, wherein the at least one peroxide activator comprises, consists essentially of or consists of at least one peroxide activator selected from the group consisting of metal-containing peroxide activators, carbonate salts and combinations thereof.

7. The method of claim 5, wherein the composition is comprised of from about 0.001% to about 20% by weight or from about 0.001% to about 5% by weight peroxide activator.

8. The method of claim 1, wherein the composition further comprises at least one thickener selected from the group consisting of viscosifying agents, gelling agents and mixtures thereof.

9. The method of claim 8, wherein the composition is comprised of from about 0.01% to about 10.0% by weight or from about 0.1% to about 5.0% by weight in total of said thickener.

10. The method of claim 1 wherein the pathogens are selected from the group consisting of the American Foulbrood (AFB) disease, Chalkbrood disease and Nosema disease.

11. The method of claim 1 wherein the pathogen is selected from the group consisting of American foulbrood (AFB), and European foulbrood (EFB); fungal diseases selected from the group consisting of Chalkbrood, Stonebrood, and Nosema; and viral diseases selected from the group consisting of Cripaviridae, Chronic bee paralysis virus, Dicistroviridae, Acute bee paralysis virus, Israeli acute paralysis virus, Kashmir bee virus, Black queen cell virus, Cloudy wing virus, Sacbrood virus; Iflaviridae—Deformed wing virus, Kakugo virus; Iridoviridae—Invertebrate iridescent virus type 6, Secoviridae—Tobacco ringspot virus, and Lake Sinai virus.

12. The method of claim 1 wherein the pathogen is bacteria selected from the group consisting of Melissococcus plutonius, Paenibacillus larvae, Spiroplasma apis, S. melliferum, Pseudomonas aeruginosa, Achromobacter euridice, Enterococcus faecalis, Paenibacillus alvei, and Brevibacillus laterosporus.

13. The method of claim 1 wherein the pathogen is fungi selected from the group consisting of Nosema apis, Nosema ceranae, Ascosphaera apis, and Aspergillus spp.

14. The method of claim 1 wherein the pathogen is responsible for a disease is a virus.

15. The method of claim 1 wherein the pathogen is a virus selected from the group consisting of Israeli acute paralysis virus, acute bee paralysis virus, Kashmir bee virus, black queen cell virus, deformed wing virus/Kakugo virus, Varroa destructor virus, sacbrood virus slow bee paralysis virus, chronic bee paralysis virus and Lake Sinai virus.

16. The method of claim 1 wherein the pathogen is selected from the group consisting of bacteria spores, fungi spores or yeast.

17. The method of claim 1 wherein the pathogen is selected from the group consisting of Varroa mites (Varroa destructor or Varroa jacobsoni), Acarine (tracheal) mites (Acarapis woodi), small hive beetles (Aethina tumida), wax moths (Galleria mellonella), and tropilaelaps (Tropilaelaps clareae and T. mercedesae)

18. The method of claim 1 wherein said composition is a bee-ingestible composition in liquid form.

19. The method of claim 1 wherein said composition is a bee-ingestible composition in solid form.

20. The method of claim 1 wherein said composition contains protein.

21. The method of claim 19 wherein said composition is based on pollen and/or soy patties.

22. The method of claim 1 wherein said composition is a bee-ingestible composition based on sucrose solution.

23. The method of claim 1 wherein said composition is a bee-ingestible composition based on corn syrup solution.

24. The method of claim 1 wherein said composition is a bee-ingestible composition that further comprises a carbohydrate.

25. The method of claim 1 wherein said composition is a bee-ingestible composition that further comprises a sugar supplement.

26. The method of claim 1 wherein the composition is administered to the bees via a device selected from the group consisting of strip, controlled release strip, tablet, reservoir, polymer disc, evaporation device, fiber, tube, polymeric block, membrane, pellet, and microcapillary comprising the composition.
